(19) United States
(12) Patent Application Publication
(10) Pub. No.: US 2026/0000375 A1
(43) Pub. Date: Jan. 1, 2026

(54) GANTRY FOR A COMPUTED TOMOGRAPHY DEVICE AND METHOD FOR EXECUTING A TRANSLATIONAL MOVEMENT IN A GANTRY

(71) Applicant: Siemens Healthineers AG, Forchheim (DE)

(72) Inventor: Florian KIESSLING, Forchheim (DE)

(73) Assignee: Siemens Healthineers AG, Forchheim (DE)

(21) Appl. No.: 19/244,163

(22) Filed: Jun. 20, 2025

(30) Foreign Application Priority Data
Jun. 28, 2024 (DE) 10 2024 206 036.4

(51) Int. Cl. A61B 6/00

(52) U.S. Cl. CPC A61B 6/4476

(57) ABSTRACT

One or more example embodiments relates to a gantry for a computed tomography device, the gantry comprising an opening forming a tunnel extending along a system axis of the gantry; a first gantry part including an X-ray source configured to produce X-ray radiation, the first gantry part annularly surrounding the opening; a second gantry part supporting the first gantry part; a linear guide, the first gantry part is movably mounted via the linear guide such that a translational movement of the first gantry part relative to the second gantry part is executable along the system axis; a threaded spindle, the threaded spindle is rotatably mounted about a spindle rotation axis via a spindle rotary bearing relative to the second gantry part, the spindle rotation axis is parallel to the system axis; a screw drive, the screw drive is configured to convert a rotational movement of the threaded spindle.

### CROSS-REFERENCE TO RELATED APPLICATION(S)

The present application claims priority under 35 U.S.C. § 119 to German Patent Application No. 10 2024 206 036.4, filed Jun. 28, 2024, the entire contents of which are incorporated herein by reference.

### FIELD

One or more example embodiments relates to a gantry for a computed tomography device. One or more example embodiments further relates to a computed tomography device and a method for executing a translational movement in a gantry.

### RELATED ART

A scanning movement, for example, can be performed, in order to examine an examination subject using a computed tomography device. The examination subject and a projection data acquisition system of the computed tomography device are moved translationally relative to each other, while the projection data from an examined region of the examination subject is captured via the projection data acquisition system, particularly in order to be able to produce a multi-layer medical image dataset.

In specific situations it can be advantageous if the scanning movement can be performed while the examination subject is stationary relative to a surrounding area of the computed tomography device, in particular relative to an examination space. To achieve this, the projection data acquisition system of the computed tomography device is moved translationally relative to the surrounding area of the computed tomography device, while the projection data from the examined region of the examination subject is captured via the projection data acquisition system.

For example, a gantry part of the computed tomography device that has the projection data acquisition system can be moved translationally relative to a gantry part of the computed tomography device that is stationary relative to the examination subject and relative to the surrounding area of the computed tomography device.

US 2024/0108294 A1 and US 2024/0108295 A1 are cited here as related art.

### SUMMARY

One or more example embodiments provides an alternative to conventional solutions for translational movement in a gantry of a computed tomography device. Each subject matter of an independent claim achieves this. Other advantageous aspects are incorporated in the dependent claims. Independent of the grammatical term usage, individuals with male, female or other gender identities are included within the term.

### BRIEF DESCRIPTION OF THE DRAWINGS

Features of example embodiments are explained below via examples with reference to the attached figures. The representation in the figures is schematic, heavily simplified, and not necessarily true to scale.

FIG. 1 shows a computed tomography device according to one or more example embodiments.

FIG. 2 shows a rear view of a gantry of a computed tomography device according to one or more example embodiments.

FIG. 3 shows a top view of a first gantry part and a second gantry part according to one or more example embodiments.

FIG. 4 shows a side view of a first gantry part and a second gantry part according to one or more example embodiments.

FIG. 5 shows a flow chart of a method for executing a translational movement in a gantry according to one or more example embodiments.

### DETAILED DESCRIPTION

One or more example embodiments relates to a gantry for a computed tomography device,
wherein the gantry has an opening, a first gantry part, a second gantry part, a linear guide, a threaded spindle, a screw drive, and a motor,
wherein the opening forms a tunnel extending along the system axis of the gantry in such a way that an examination subject can be introduced into the opening along the system axis and can be examined in the opening via X-ray radiation,
wherein the first gantry part annularly surrounds the opening and has an X-ray source for producing the X-ray radiation,
wherein the second gantry part is set up to support the first gantry part, wherein the first gantry part is movably mounted via the linear guide in such a way relative to the second gantry part that a translational movement of the first gantry part relative to the second gantry part can be executed along the system axis,
wherein the threaded spindle is rotatably mounted about a spindle rotation axis via a spindle rotary bearing relative to the second gantry part, wherein the spindle rotation axis is parallel to the system axis,
wherein the screw drive is set up to convert a rotational movement of the threaded spindle relative to the second gantry part about the spindle rotation axis into the translational movement of the first gantry part relative to the second gantry part along the system axis,
wherein the motor is set up to drive the rotational movement of the threaded spindle.

In particular, it can be provided that the system axis is horizontal and/or that the spindle rotation axis is horizontal. In particular, it can be provided that the translational movement of the first gantry part relative to the second gantry part occurs parallel to the system axis. The screw drive can be, for example, a trapezoidal screw drive, a ball screw drive, a roller screw drive, or a planetary screw drive, in particular a planetary roller screw drive. The examination subject can be, for example, a body part, in particular a head, of a human or an animal, or a phantom.

One embodiment provides that the first gantry part has a spindle nut of the screw drive, wherein the spindle nut works with the threaded spindle, in order to convert the rotational movement of the threaded spindle relative to the second gantry part about the spindle rotation axis into the translational movement of the first gantry part relative to the second gantry part along the system axis. In particular, it can be provided that the gantry has the spindle rotary bearing.

One embodiment provides that the spindle rotary bearing comprises a locating bearing. In particular, it can be provided that the locating bearing is located in relation to the spindle rotation axis between the motor and the screw drive, in particular between the motor and the spindle nut. The locating bearing can be, for example, an angular ball bearing and/or a four-point bearing. The angular ball bearing can be, for example, a two-row angular ball bearing or a combination of two one-row angular ball bearings.

One embodiment provides that the spindle rotary bearing comprises a floating bearing. In particular, it can be provided that the screw drive and/or the spindle nut are located in relation to the spindle rotation axis between the locating bearing and the floating bearing. The floating bearing can be, for example, a deep-groove ball bearing. The threaded spindle is thus able to expand and/or contract due to temperature changes without causing unwanted mechanical tensions and/or deformations as a result.

One embodiment provides that the motor is coupled to the threaded spindle via a claw coupling, in particular with torsional elasticity. In particular, it can be provided that the claw coupling is located in relation to the spindle rotation axis between the motor and the locating bearing and/or that the locating bearing is located in relation to the spindle rotation axis between the claw coupling and the screw drive, in particular between the claw coupling and the spindle nut.

In particular, it can be provided that a motor shaft of the motor is coaxial to the spindle rotation axis. In particular, it can be provided that the claw coupling has an elastomer to compensate for alignment errors. Thus, for example, misalignment between the threaded spindle and a motor shaft of the motor can be compensated, particularly lateral, angular, and/or axial misalignment. Furthermore, impacts and/or fluctuations can thus be damped, for example. The claw coupling can be, for example, open or closed. In particular, it can be provided that the motor, via the claw coupling, for example, is coupled to the threaded spindle directly and/or with a speed transmission ratio of one to one. Moreover, the noise level can be minimized with a suitable choice of drive control, so that no additional sound reductions or decoupling measures are necessary.

One embodiment provides that the motor is a stepper motor. In contrast to a direct current drive, in the form of a brushed DC motor or in the form of a brushless EC motor, for example, a stepper motor can be operated at the same torque at lower speeds, so that speed transmission to slow is not necessary. Thus, both the space requirements and the complexity of the mechanism can be cost-effectively reduced.

One embodiment provides that the gantry has a rotary encoder for capturing rotary encoder measurement data, wherein the rotary encoder measurement data relates to a rotation angle and/or rotation angle change of the threaded spindle about the spindle rotation axis. In particular, it can be provided that the motor is located in relation to the spindle rotation axis between the rotary encoder and the locating bearing and/or between the rotary encoder and the claw coupling.

One embodiment provides that the gantry has a control unit, wherein the control unit is set up for field-oriented control of the rotational movement of the threaded spindle. Field-oriented control can be effected, for example, on the basis of the rotary encoder measurement data and/or on the basis of positioning information relating to a target position of the first gantry part relative to the second gantry part along the system axis; in particular, field-oriented control can be effected in such a way that the translational movement of the first gantry part relative to the second gantry part along the system axis causes the first gantry part to be in the target position relative to the second gantry part along the system axis.

The control unit can have, for example, at least one field-programmable gate array (FPGA). The field-oriented control can be implemented in particular in the FPGA. Accordingly, it is possible to achieve a high-speed translational movement on the one hand and highly accurate positioning on the other.

One embodiment provides that the linear guide has a rail system and a carriage system, which works with (cooperates with) the rail system. One embodiment provides that the first gantry part has the carriage system and that the second gantry part has the rail system.

One embodiment provides that the first gantry part has an X-ray detector for detecting the X-ray radiation. In particular, it can be provided that the first gantry part has a projection data acquisition system for capturing projection data. The projection data acquisition system can, for example, have the X-ray source and/or the X-ray detector.

In particular, it can be provided that the first gantry part has a rotor, a rotary bearing, and a support frame, wherein the rotor is connected to the support frame via the rotary bearing and is rotatably mounted about the system axis relative to the support frame. In particular, it can be provided that the rotor has the X-ray source and/or the X-ray detector and/or that the rotor has the projection data acquisition system. The support frame can, for example, have the carriage system.

One embodiment provides that the first gantry part has a rear side of a gantry cladding, wherein the rear side of the gantry cladding annularly surrounds a rear side of the opening. In particular, it can be provided that the gantry has a third gantry part, wherein the third gantry part has a front side of the gantry cladding, wherein the front side of the gantry cladding annularly surrounds a front side of the opening. In particular, it can be provided that, at least in one operating state of the gantry, the first gantry part is movably mounted in such a way relative to the third gantry part that the translational movement of the first gantry part relative to the second gantry part can be executed along the system axis, while the third gantry part is stationary relative to the second gantry part. In particular, it can be provided that the third gantry part annularly surrounds the opening.

One embodiment provides that the second gantry part has a chassis, wherein the gantry is movably mounted relative to a base area via the chassis, in particular is movably mounted horizontally. In particular, it can be provided that the chassis is wheel-based and/or rail-based and/or that the base area is essentially horizontal.

One or more example embodiments further relates to a computed tomography device having the gantry according to one or more example embodiments. The computed tomography device can be in particular a mobile computed tomography device and/or be set up for head imaging.

One or more example embodiments further relates to a method for executing a translational movement in a gantry according to one or more example embodiments, the method comprising:
driving the rotational movement of the threaded spindle relative to the second gantry part about the spindle rotation axis via the motor,
executing the translational movement of the first gantry part relative to the second gantry part, by converting the rotational movement of the threaded spindle via the screw drive relative to the second gantry part about the spindle rotation axis into the translational movement of the first gantry part relative to the second gantry part along the system axis.

Within the scope of the invention, features that are described in relation to different embodiments and/or different claim categories (method, use, apparatus, system, arrangement, etc.) can be combined with other embodiments.

For example, a claim that relates to an apparatus can also be developed by features that are described or claimed in connection with a method and vice versa. Functional features of a method can be executed by correspondingly embodied representative components. Use of the indefinite article “a” or “an” does not prevent the feature concerned being present multiple times. The expression “on the basis of” can be understood in the context of the present application in particular as “using.”

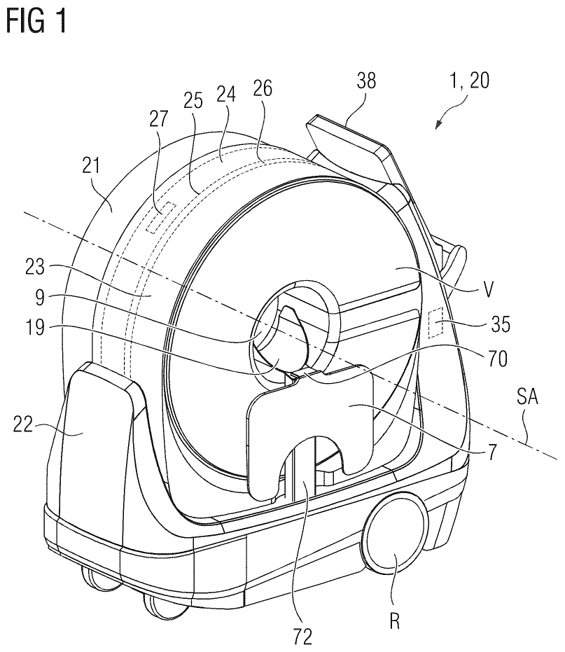

FIG. 1 shows a computed tomography device 1 in the form of a mobile head computed tomography device with a gantry 20,
wherein the gantry 20 has an opening 9, a first gantry part 21, a second gantry part 22, a linear guide, a threaded spindle 51, a screw drive 53, and a motor 56,
wherein the opening 9 forms a tunnel extending along the system axis SA of the gantry 20 in such a way that an examination subject 14 can be introduced into the opening 9 along the system axis SA and can be examined in the opening 9 via X-ray radiation 7,
wherein the first gantry part 21 annularly surrounds the opening 9 and has an X-ray source 7A for producing the X-ray radiation 7,
wherein the second gantry part 22 is set up to support the first gantry part 21, wherein the first gantry part 21 is movably mounted via the linear guide in such a way relative to the second gantry part 22 that a translational movement of the first gantry part 21 relative to the second gantry part 22 can be executed along the system axis SA,
wherein the threaded spindle 51 is rotatably mounted about a spindle rotation axis 5A via a spindle rotary bearing relative to the second gantry part 22, wherein the spindle rotation axis 5A is parallel to the system axis SA,
wherein the screw drive 53 is set up to convert a rotational movement of the threaded spindle 51 relative to the second gantry part 22 about the spindle rotation axis 5A into the translational movement of the first gantry part 21 relative to the second gantry part 22 along the system axis SA,
wherein the motor 56 is set up to drive the rotational movement of the threaded spindle 51.

The second gantry part 22 has the touch-sensitive screen 38 for controlling the computed tomography device 1. The second gantry part 22 has the holding apparatus 72, the head shell 19 for the person's head, and the body support device 7 to support the person's body relative to the opening 9. The holding apparatus 72 extends along the vertical direction Y. The shoulder board 7 is connected to the holding apparatus 72 via the pivot device 70 and is pivotally mounted about a pivot axis perpendicular to the system axis SA relative to the gantry 20. The system axis SA is horizontal and parallel to the direction Z and parallel to the direction Z1. The horizontal direction X is perpendicular to the system axis SA.

The illustrated example shows that the first gantry part 21 has a rear side of a cladding V of the gantry 20, wherein the rear side of the cladding V of the gantry 20 annularly surrounds a rear side of the opening 9. In particular, it is provided that the gantry 20 has a third gantry part 23, wherein the third gantry part 23 has a front side of the cladding V of the gantry 20, wherein the front side of the cladding V of the gantry 20 annularly surrounds a front side of the opening 9. In particular, it is provided that the first gantry part 21, at least in one operating state of the gantry 20, is movably mounted in such a way relative to the third gantry part 23 that the translational movement of the first gantry part 21 relative to the second gantry part 22 can be executed along the system axis SA, while the third gantry part 23 is stationary relative to the second gantry part 22. In particular, it is provided that the third gantry part 23 annularly surrounds the opening 9.

The illustrated example provides that the second gantry part 22 has a chassis R, wherein the gantry 20 is movably mounted via the chassis R relative to a base area.

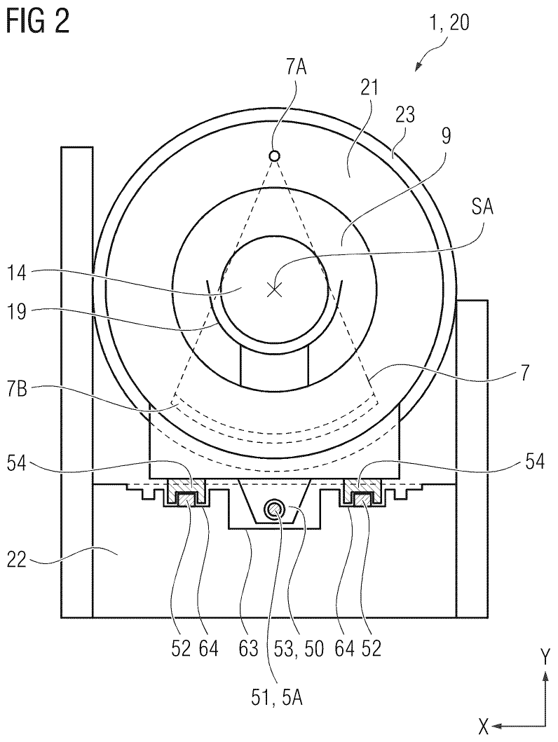

FIG. 2 shows a rear view of the gantry 20 of the computed tomography device 1. The second gantry part 22 has the indentations 64 for the linear guide and an indentation 63 for the threaded spindle 51 and the screw drive 53. The illustrated example provides that the first gantry part 21 has a spindle nut 50 of the screw drive 53, wherein the spindle nut 50 works with the threaded spindle 51, in order to convert the rotational movement of the threaded spindle 51 relative to the second gantry part 22 about the spindle rotation axis 5A into the translational movement of the first gantry part 21 relative to the second gantry part 22 along the system axis SA.

The illustrated example shows that the linear guide has a rail system 52 and a carriage system 54, which works with the rail system 52. The illustrated example provides that the first gantry part 21 has the carriage system 54 and that the second gantry part 22 has the rail system 52. The illustrated example provides that the first gantry part 21 has an X-ray detector 7B for detecting the X-ray radiation 7.

In particular, it is provided that the first gantry part 21 has a projection data acquisition system 27 for capturing projection data. The projection data acquisition system 27 can have, for example, the X-ray source 7A and/or the X-ray detector 7B. In particular, it is provided that the first gantry part 21 has a rotor 24, a rotary bearing 25, and a support frame 26, wherein the rotor 24 is connected to the support frame 26 via the rotary bearing 25 and is rotatably mounted about the system axis SA relative to the support frame 26. In particular, it is provided that the rotor 24 has the X-ray source 7A and/or the X-ray detector 7B and/or that the rotor 24 has the projection data acquisition system 27. The support frame 26 can, for example, have the carriage system 54.

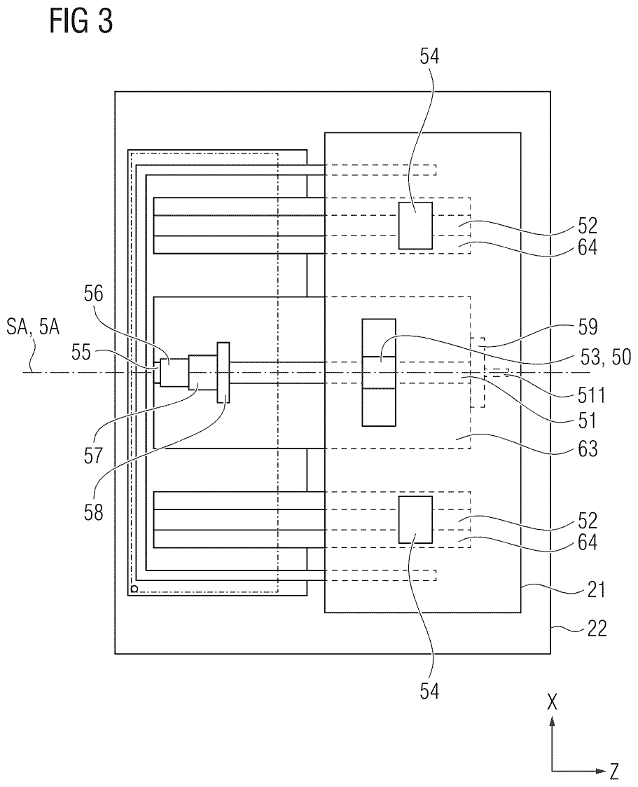

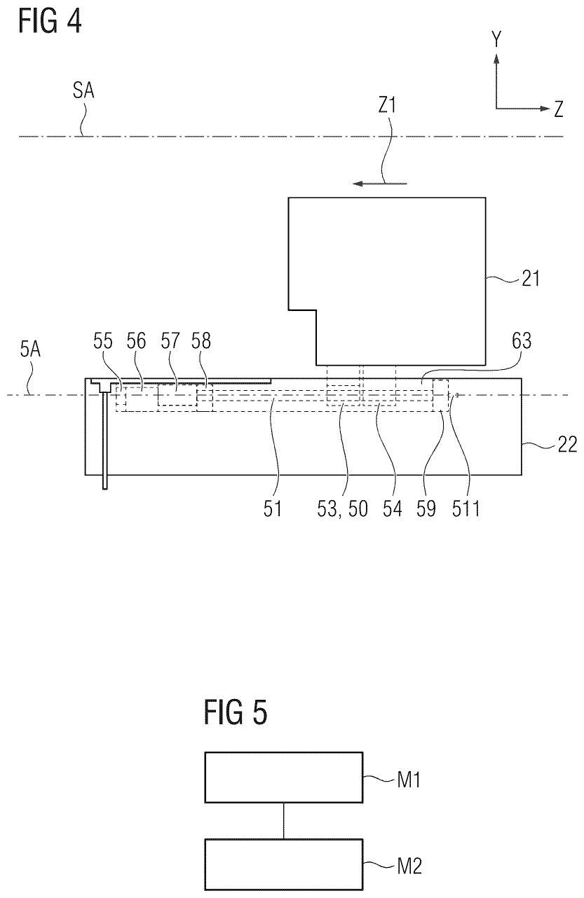

FIG. 3 shows a top view of the first gantry part 21 and the second gantry part 22. FIG. 4 shows a side view of the first gantry part 21 and the second gantry part 22. The axial extension 511 forms an interface for attaching a screwdriver or wrench. The interface can have, for example, a screw-head profile, particularly in the form of a hexagon socket or hexagon head, which is rigidly connected to the threaded spindle 51.

The illustrated example provides that the spindle rotary bearing comprises a locating bearing 58. In particular, it is provided that the locating bearing 58 is located in relation to the spindle rotation axis 5A between the motor 56 and the screw drive 53, in particular between the motor 56 and the spindle nut 50. The illustrated example provides that the spindle rotary bearing comprises a floating bearing 59. In particular, it is provided that the screw drive 53 and/or the spindle nut 50 are located in relation to the spindle rotation axis 5A between the locating bearing 58 and the floating bearing 59.

The illustrated example provides that the motor 56 is coupled via a claw coupling 57 to the threaded spindle 51. In particular, it is provided that the claw coupling 57 is located in relation to the spindle rotation axis 5A between the motor 56 and the locating bearing 58 and that the locating bearing 58 is located in relation to the spindle rotation axis 5A between the claw coupling 57 and the screw drive 53, in particular between the claw coupling 57 and the spindle nut 50.

The illustrated example provides that the motor 56 is a stepper motor. The illustrated example provides that the gantry 20 has a rotary encoder 55 for capturing rotary encoder measurement data, wherein the rotary encoder measurement data relates to a rotation angle and/or rotation angle change of the threaded spindle 51 about the spindle rotation axis 5A. In particular, it is provided that the motor 56 is located in relation to the spindle rotation axis 5A between the rotary encoder 55 and the locating bearing 58 and/or between the rotary encoder 55 and the claw coupling 57. The illustrated example provides that the gantry 20 has a control unit 35, wherein the control unit 35 is set up for field-oriented control of the rotational movement of the threaded spindle 51.

FIG. 5 shows a flow chart of a method for executing a translational movement in the gantry 20, the method comprising:
driving M1 the rotational movement of the threaded spindle 51 relative to the second gantry part 22 about the spindle rotation axis 5A via the motor 56,
executing M2 the translational movement of the first gantry part 21 relative to the second gantry part 22, by converting the rotational movement of the threaded spindle 51 via the screw drive 53 relative to the second gantry part 22 about the spindle rotation axis 5A into the translational movement of the first gantry part 21 relative to the second gantry part 22 along the system axis SA.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being transferred, stored, combined, compared, and otherwise manipulated. It has proven convenient at times, principally for reasons of common usage, to refer to these signals as bits, values, elements, symbols, characters, terms, numbers, or the like.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Although the present invention has been described in accordance with preferred embodiments, it is obvious for the person skilled in the art that modifications are possible in all embodiments.

1. A gantry for a computed tomography device, the gantry comprising:
an opening, the opening forming a tunnel extending along a system axis of the gantry such that an examination subject being introducible into the opening along the system axis and being examinable in the opening via X-ray radiation;
a first gantry part including an X-ray source configured to produce the X-ray radiation, the first gantry part annularly surrounding the opening;
a second gantry part, the second gantry part supporting the first gantry part;
a linear guide, the first gantry part is movably mounted via the linear guide such that a translational movement of the first gantry part relative to the second gantry part being executable along the system axis;
a threaded spindle, the threaded spindle is rotatably mounted about a spindle rotation axis via a spindle rotary bearing relative to the second gantry part, the spindle rotation axis being parallel to the system axis;
a screw drive, the screw drive being configured to convert a rotational movement of the threaded spindle relative to the second gantry part about the spindle rotation axis into the translational movement of the first gantry part relative to the second gantry part along the system axis; and
a motor configured to drive the rotational movement of the threaded spindle.

2. The gantry of claim 1, wherein
the first gantry part includes a spindle nut of the screw drive, and
the spindle nut works with the threaded spindle to convert the rotational movement of the threaded spindle relative to the second gantry part about the spindle rotation axis into the translational movement of the first gantry part relative to the second gantry part along the system axis.

3. The gantry of claim 1, wherein the spindle rotary bearing comprises a locating bearing.

4. The gantry of claim 1, wherein the spindle rotary bearing comprises a floating bearing.

5. The gantry of claim 1, wherein the motor is coupled via a claw coupling to the threaded spindle.

6. The gantry of claim 1, wherein the motor is a stepper motor.

7. The gantry of claim 1, wherein the gantry includes a rotary encoder configured to capture rotary encoder measurement data, and the rotary encoder measurement data relates to at least one of a rotation angle of the threaded spindle about the spindle rotation axis or a rotation angle change of the threaded spindle about the spindle rotation axis.

8. The gantry of claim 1, wherein
the gantry includes a control unit, the control unit is configured for field-oriented control of the rotational movement of the threaded spindle.

9. The gantry of claim 1, wherein the linear guide includes a rail system and a carriage system, the carriage system cooperates with the rail system.

10. The gantry of claim 1, wherein the first gantry part includes a carriage system and the second gantry part includes a rail system, the carriage system cooperates with the rail system.

11. The gantry of claim 1, wherein the first gantry part includes an X-ray detector configured to detect the X-ray radiation.

12. The gantry of claim 1, wherein the first gantry part includes a rear side of a cladding of the gantry, the rear side of the cladding of the gantry annularly surrounds a rear side of the opening.

13. The gantry of claim 1, wherein the second gantry part includes a chassis, the gantry is movably mounted via the chassis relative to a base area.

14. A computed tomography device comprising:
the gantry of claim 1.

15. A method for executing a translational movement in the gantry of claim 1, the method comprising:
driving the rotational movement of the threaded spindle relative to the second gantry part about the spindle rotation axis via the motor; and
executing the translational movement of the first gantry part relative to the second gantry part, by converting the rotational movement of the threaded spindle via the screw drive relative to the second gantry part about the spindle rotation axis into the translational movement of the first gantry part relative to the second gantry part along the system axis.

16. The gantry of claim 2, wherein the motor is a stepper motor.

17. The gantry of claim 16, wherein the gantry includes a rotary encoder configured to capture rotary encoder measurement data, and the rotary encoder measurement data relates to at least one of a rotation angle of the threaded spindle about the spindle rotation axis or a rotation angle change of the threaded spindle about the spindle rotation axis.

18. The gantry of claim 17, wherein
the gantry includes a control unit, the control unit is configured for field-oriented control of the rotational movement of the threaded spindle.

19. The gantry of claim 18, wherein the linear guide includes a rail system and a carriage system, the carriage system cooperates with the rail system.

20. The gantry of claim 18, wherein the first gantry part includes a carriage system and the second gantry part includes a rail system, the carriage system cooperates with the rail system.
